(19) United States
(12) Patent Application Publication
(10) Pub. No.: US 2022/0067802 A1
(43) Pub. Date: Mar. 3, 2022

(54) Calculation of patient minimum payment due based on propensity to default score .

(71) Applicant: AKHIL AGGARWAL, DAYTON, OH (US)

(72) Inventor: AKHIL AGGARWAL, DAYTON, OH (US)

(21) Appl. No.: 17/003,934

(22) Filed: Aug. 26, 2020

(51) Int. Cl. G06Q 30/04; G06Q 40/08; G06Q 10/10; G16H 10/60

(52) U.S. Cl. CPC G06Q 30/04; G16H 10/60; G06Q 10/10; G06Q 40/08

(57) ABSTRACT

A computerized method for calculating a patient's propensity to default on a medical bill balance and the amount due at the time of a doctor's visit in order to keep the patient's account in good standing. The computerized method allows the medical practice to set how much time it wants to allow patients to pay off the balance on a medical claim (e.g. 180 days). A computer program calculates and provides an amount due to a user of the computer program but the user can override the provided amount due and accept a lower payment amount from the patient. The computer program user needs to add notes if he/he chooses to override the amount calculated and provided by the computer program. All of the data related to the override is logged along with details related to the user, and the notes etc. so that the management of the medical facility can review user activity.

### FIELD OF THE INVENTION

The invention relates to medical bill payment.

### BACKGROUND OF THE INVENTION

When a patient visits a doctor's office (often referred to as a medical practice) the whole visit starts with a check-in procedure at the reception desk. The check-in process mainly comprises of checking whether the patient has an appointment today, the insurance eligibility (and any co-pay) for the patient, and last but not the least, whether there is any patient balance currently owed to the medical practice by the patient. This last element of the check-in process where human interaction comes into play. The other 2 components of the check-in process are pretty straightforward. It is the patient balance in current the environment of sky rocketing patients deductibles that has somehow become an essential part of patient care. The patient usually has several reasons for not being able to maintain his/her account in good standing (zero balance). Determining how much a patient should pay on balance currently owed is quite arbitrary . . . the reception staff may not even realize that patient payment today will just cover the co-pay for the services provided today. This results in the patient piling up a huge medical bill balance which eventually gets turned over to a collection agency, ruining their credit, chance to get a job, and so on.

### SUMMARY OF THE INVENTION

The invention comprises-of a formula to calculate a patient's propensity to default on a balance (based on past payments) and calculation of a minimum payment amount due on a given day based on the patent propensity to default score. The patent propensity to default score tells the medical practice as to how likely is patient to not pay on his/her balance. This will result in eradication of arbitrary payment amounts or in other words will lead to standardization of how a medical practice comes up with minimum patient payment amount due on a visit. This also leads to consideration of every patient as equal and a better understanding by the patient of their financial responsibility as far as their medical bill is concerned, instead of getting surprised later on.

### BRIEF DESCRIPTION OF THE DRAWINGS

These and further aspects of the invention will become apparent from the following detailed description.

FIG. 1—depicts retrieval of data from an Electronic Health Record (EHR) system by the invented computer program through means of Application Program Interface API) calls.

FIG. 2 is a Datasheet 1 which depicts the calculation of patient propensity to default score (Stage 1), or in other words how likely is a patient is to not pay on a balance owed.

FIG. 3 is a Datasheet 2 which depicts the calculation of minimum payment due on a visit (Stage 2) based on the patient propensity to default score.

### DETAILED DESCRIPTION OF THE INVENTION

The invention is based on one major component, the default number of days a practice allows it patients to pay-off a medical claim. For example, if a patient has to pay a deductible of $100 and the practice has set the default number of days to pay off a medical claim at 120 days, this means the patient has 120 days to pay the $100 balance from the day the balance is posted for the respective claim. The invented program is a two-step calculation, first a patient propensity to default score is calculated and second a minimum payment amount due is calculated using the patent propensity to default score. The key components required to calculate these two numbers are as following:

1. Default number of days allowed by the practice to pay_off a medical claim (e.g. 120 days)

2. Patient balance on a claim (e.g. $100)

3. Date of patient balance posting on a claim (e.g. 1/27/19)

4. Patient payments on a claim (e.g. $25; $25 etc.)

5. Date(s) of patient payment on a claim (e.g. 2/14/19; 3/14/19 etc.)

6. Days to full payment on a claim.

7. Today's date (date for which minimum payment amount needs to be calculated.)

8. Charge score (explained below)

The invention is intended to be used with a Electronic Health record (EHR/EMR) system. The intent of the invention is to standardize the process of calculating minimum payment due on patent balance.

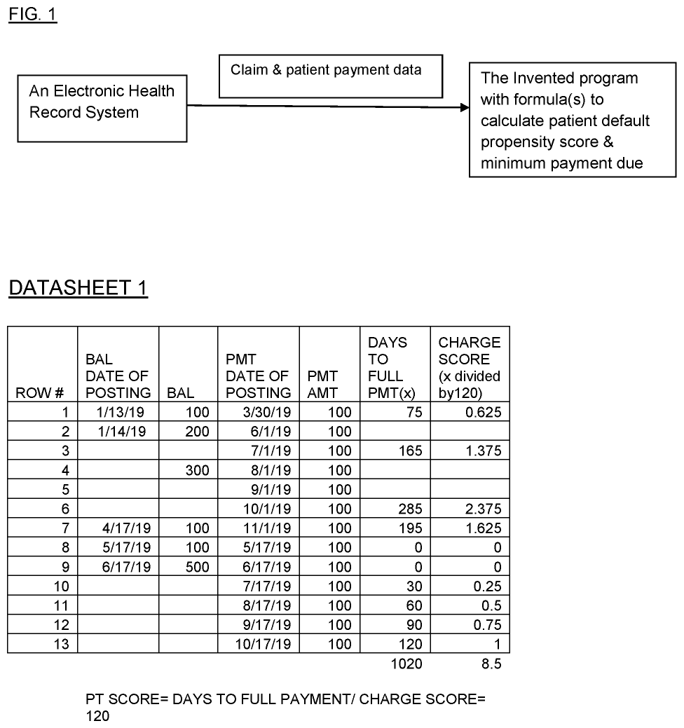

As shown in FIG. 1, the computer program will make Application Interface (API) call(s) to the EHR to get-data; as shown. The data is used by the computer program in a first stage to electronically calculate the default propensity score. First, the actual number of days to when full payment was made is calculated for each of the prior medical claims of a patient , wherein (the number of days to full payment) equals (the full payment date) minus (the date of balance posting). For example, if the date of the balance posting is 05/25/2018 and the date of full payment of the balance posting is 07/04/2019, the actual number of days to full payment is 07/04/2019-05/25/2018=40 days. Next, the actual number of days to full payment is utilized to electronically calculate a charge score for each of the prior medical claims of the patient, wherein (the charge score) equals (the actual number of days to full payment) divided by (the default number of days set by the medical practice for full payment). For example, the medical practice sets the default number of days for full payment at 120 days, the charge score is 40/120=0.33. Finally, the charge score is used to electronically calculate a patient propensity to default score, wherein (the patient propensity to default score) equals (the sum of each of the actual number of days to full payment) divided by (the sum of each of the charge scores).

In a second stage, the computer program calculates the minimum payment amount expected from a patient on a given day based on the patient's propensity to default score.

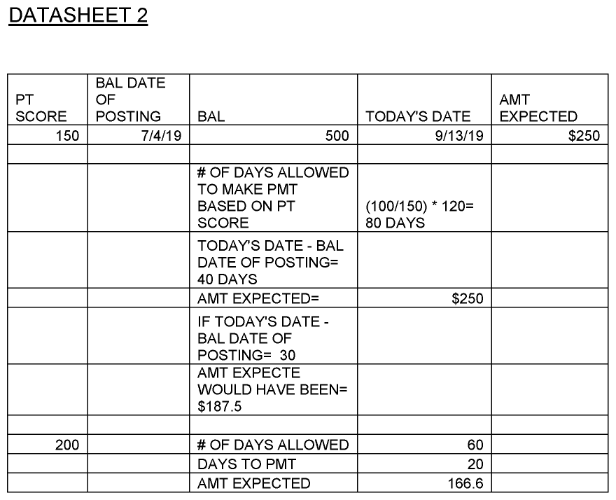

As shown in FIG. 2, Datasheet 1 illustrates the scenario where a patient balance is posted after a claim is processed by medical insurance company and patient makes payments thereafter. Based on these payments, a patient propensity to default score is being calculated, this can also be called patient score.

Datasheet 2 The second data sheet focuses on calculation of minimum payment based on the patient propensity to default score (Calculated in Datasheet 1).

The computer program calculates and provides an amount due to a user of the computer program but the user can override the provided amount due and accept a lower payment amount from the patient. The computer program user needs to add notes if he/he chooses to override the amount calculated and provided by the computer program. All of the data related to the override is logged along with details related to the user, and the notes etc. so that the management of the medical facility can review user activity.

1. A computer implemented method, implemented on an information security computing machine, for securely calculating a minimum payment amount due for a patient at a medical facility, the method comprising the steps of:
automatically and electronically, running application program interface calls, by the information security machine, to an electronic health records system to obtain each balance due posting date for-a prior medical claims of the patient and each full payment date for the prior medical claims of the patient;
obtaining a default number of days allowed to pay off medical claims set by the medical facility and providing the default number of days allowed to pay off medical claims to the information security machine;
automatically and electronically calculating, by the information security machine, an actual number of days to full payment for each of the prior medical claims of the patient, wherein the actual number of days to full payment=(the full payment date for the prior medical claim)−(the balance due posting date for the prior medical claim);
automatically and electronically calculating, by the information security machine, a charge score for each of the prior medical claims for the patient, wherein the charge score=(the actual number of days to full payment for each of the prior medical claims)/(the default number of days allowed to pay off medical claims);
automatically and electronically calculating, by the information security machine, a default propensity score for the patient, wherein the default propensity score=(the sum the actual number of days to full payment for each of the prior medical claims)/(the sum of the sum of the charge scores for each of prior medical claims;
automatically and electronically calculating, by the information security machine, a minimum payment amount due for the patient based on the calculated default payment propensity score for the patient; and
automatically and electronically providing by the information security machine, an electronic notification of the minimum payment amount due for the patient to the user.
